(19) United States
(12) Patent Application Publication
(10) Pub. No.: US 2012/0288443 A1
(43) Pub. Date: Nov. 15, 2012

(54) LABELLED PYRROLYL-OXADIAZOLYL-DIAZABICYCLONONANE DERIVATIVES AND THEIR USE IN DIAGNOSTIC METHODS

(75) Inventors: Dan Peters, Malmo (SE); Daniel B. Timmermann, Herlev (DK); Jens Damsgaard Mikkelsen, Lyngby (DK); Anders Janusz Ettrup, Frederiksberg (DK)

(73) Assignee: NEUROSEARCH A/S, Ballerup (DK)

(21) Appl. No.: 13/509,215

(22) PCT Filed: Nov. 9, 2010
(86) PCT No.: PCT/EP10/67096

§ 371 Date: Aug. 1, 2012

Related U.S. Application Data
(60) Provisional application No. 61/261,477, filed on Nov. 16, 2009.

(30) Foreign Application Priority Data
Nov. 11, 2009 (DK) PA 2009 01206
Dec. 8, 2009 (DK) PA 2009 01293
Mar. 16, 2010 (DK) PA 2010 70105

(51) Int. Cl. C07D 487/08; A61K 51/04

(52) U.S. Cl. 424/1.81; 540/556

(57) ABSTRACT

The present invention relates to certain labelled pyrrolyl-oxadizolyl-diazabicyclononane derivatives. Furthermore, the present invention relates to the use of said derivatives in their labelled form in diagnostic methods, in particular for in vivo receptor imaging (neuroimaging).

### TECHNICAL FIELD

The present invention relates to certain labelled pyrrolyl-oxadizolyl-diazabicyclononane derivatives. Furthermore, the present invention relates to the use of said derivatives in their labelled form in diagnostic methods, in particular for in vivo receptor imaging (neuroimaging).

### BACKGROUND ART

Neuroimaging is the use of certain technologies to measure a brain function or an aspect related to the functioning of certain parts of the brain, and enables the processing of information by centers in the brain to be visualized directly. Neuroimaging often requires the use of radioligands which have desirable properties for in vivo receptor imaging. These criteria include ease of labelling with positron-emitting radionucleotides, low rates of peripheral metabolism, high selectivity for brain regions holding the neuroreceptor of interest, and relatively high specific/non-specific binding ratios.

WO 2004/029053, WO 2007/138037 and WO 2007/138038 all describes oxadiazolyl-diazabicyclononane derivatives, which are found to be cholinergic ligands at the nicotinic acetylcholine receptors and modulators of the monoamine receptors and transporters. However, the labelled pyrrolyl-oxadiazole-diazabicyclononane derivatives of the present invention are not reported.

### SUMMARY OF THE INVENTION

It is an object of the present invention to provide a compound that can be used as a radiotracer or radio-labelled substance to monitor in vivo and in vitro levels of nicotinic acetylcholine receptors, and in particular the nicotinic α7 receptor subtype, by way of non-invasive determination of the localisation of such receptors (neuroimaging).

This object is solved by providing an labelled pyrrolyl-oxadiazole-diazabicyclononane derivative represented by Formula I

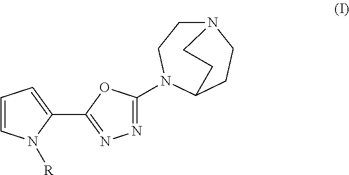

or a pharmaceutically acceptable salt thereof, wherein R represents a labelled C₁₋₆-alkyl group.

In another aspect, the invention provides pharmaceutical compositions comprising a diagnostically effective amount of a labelled pyrrolyl-oxadiazole-diazabicyclononane derivative of the invention, or a pharmaceutically acceptable addition salt thereof, together with at least one pharmaceutically acceptable carrier or diluent.

In a further aspect the invention provides methods for the non-invasive determination of the distribution of a tracer compound inside a whole, intact living animal or human body using a physical detection method, wherein the tracer compound is a compound of the invention, or a pharmaceutically acceptable salt thereof.

Other objects of the invention will be apparent to the person skilled in the art from the following detailed description and examples.

### DETAILED DISCLOSURE OF THE INVENTION

Labelled pyrrolyl-oxadiazole-diazabicyclononane derivatives

In its first aspect the invention provides a labelled pyrrolyl-oxadiazole-diazabicyclononane derivative represented by Formula I

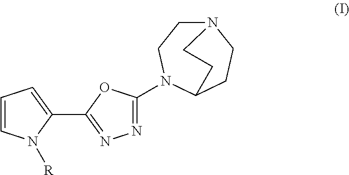

or a pharmaceutically acceptable salt thereof, wherein R represents a labelled C₁₋₆-alkyl group.

In a preferred embodiment the labelled pyrrolyl-oxadiazole-diazabicyclononane derivative of the invention is a compound of Formula I, wherein the pyrrolyl group additionally is labelled by incorporation of one or more ¹⁸F.

In another preferred embodiment the C₁₋₆-alkyl group is labelled by incorporation of one or more ¹¹C, ¹³C or ¹⁴C.

In a third preferred embodiment the C₁₋₆-alkyl group is labelled by incorporation of one or more ¹¹C.

In a fourth preferred embodiment R represents a labelled methyl or ethyl group.

In a most preferred embodiment the labelled pyrrolyl-oxadiazole-diazabicyclononane derivative of the invention is

2-(1,4-Diazabicyclo[3.2.2]nonan-4-yl)-5-(1-[¹¹C]methylpyrrol-2-yl)-1,3,4-oxadiazole;

or a pharmaceutically acceptable salt thereof.

Labelled Compounds

In the context of this invention a labelled compound has one or more atoms replaced by an atom having an atomic mass or mass number different from the atomic mass or mass number usually found in nature. Such labelling will allow easy quantitative detection of the compound in question.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹¹C, ¹³ C, ¹⁴C, ¹³¹I, ¹²⁵I, ¹²³I and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

Definition of Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

Pharmaceutically Acceptable Salts

The diazabicyclic aryl derivative of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a chemical compound of the invention include alkali metal salts, such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Preparation of Labelled pyrrolyl-oxadiazole-diazabicyclononane Derivatives

The pyrrolyl-oxadiazole-diazabicyclononane derivatives of the invention may be prepared by conventional methods for chemical synthesis, e.g. those described in the working examples.

Labelling of the 1,4-diazabicyclo[3.2.2]nonane derivative of the invention may also be accomplished in analogy with the method described by e.g. Jensen et al. [Jensen SB, Bender D, Smith D F, Scheel-Krüger J, Nielsen EØ, Olsen G M, Peters D & Gjedde A: Synthesis of (±) 3-(6-nitro-2-quinolinyl)-[9-methyl-¹¹C]-3,9-diazabicyclo-[4.2.1]-nonane; J. Label. Compd. Radiopharm. 2002 45 181-189].

The end products of the reactions described herein may be isolated by conventional techniques, e.g. by extraction, crystallisation, distillation, chromatography, etc.

Neuroimaging

The pyrrolyl-oxadiazole-diazabicyclononane derivatives of the invention are useful as diagnostic tools or monitoring agents in various diagnostic methods, and in particular for in vivo receptor imaging (neuroimaging).

In another aspect of the invention, a method for the non-invasive determination of the distribution of a tracer compound inside a whole, intact living animal or human body using a physical detection method is provided. According to this method a tracer compound is a compound of the invention, or a pharmaceutically acceptable salt thereof, in labelled form.

In a preferred embodiment the physical detection method is selected from Positron Emission Tomography (PET), Single Photon Emission Computed Tomography (SPECT), Magnetic Resonance Spectroscopy (MRS), Magnetic Resonance Imaging (MRI), Computed Axial Tomography (CAT), Computed Tomography (CT), Functional Magnetic Resonance Imaging (fMRI), or combinations thereof.

The labelled compound of the invention preferably contains at least one radionuclide as a label. Positron emitting radionuclides are all candidates for usage. In the context of this invention the radionuclide is preferably selected from ²H (deuterium), ³H (tritium), ¹¹C, ¹³C, ¹⁴C, ¹⁵O, ¹³N, ¹²³I, ¹²⁵I, ¹³¹I, ¹⁸F and ^{99m}Tc.

Examples of commercially available labelling agents, which can be used in the preparation of the labelled compounds of the present invention are [¹¹C]O₂. ¹⁸F, and Nal with different isotopes of Iodine. In particular [¹¹C]O₂ may be converted to a [¹¹C]-methylating agent, such as [¹¹C]H₃I or [¹¹C]-methyl triflate.

The tracer compound can be selected in accordance with the detection method chosen.

In one preferred embodiment, the compounds of the invention labelled by incorporation of a isotope into the molecule, which may in particular be an isotope of the naturally occurring atoms including ²H (deuterium), ³H (tritium), ¹¹C, ¹³C, ¹⁴C, ¹⁵O, ¹³N, ¹²³I, ¹²⁵I, ¹³¹I, ¹⁸F and ^{99m}Tc, and the isotope incorporation may be measured by conventional scintillation counting techniques.

In another preferred embodiment, the physical method for detecting said tracer compound of the present invention is selected from Positron Emission Tomography (PET), Single Photon Emission Computed Tomography (SPECT), Magnetic Resonance Spectroscopy (MRS), Magnetic Resonance Imaging (MRI), Computed Axial Tomography (CAT), Computed Tomography (CT), Functional Magnetic Resonance Imaging (fMRI), or combinations thereof.

In a more preferred embodiment the compound of the invention is labelled by incorporation of ¹¹C, ¹³C or ¹⁴C, and the isotope incorporation is measured by Positron Emission Tomography (PET) or Single Photon Emission Computed Tomography (SPECT).

In an even more preferred embodiment the compound of the invention is labelled by incorporation of ¹¹C, and the isotope incorporation is measured by Positron Emission Tomography (PET).

Before conducting the method of the present invention, a diagnostically effective amount of a labelled compound of the invention is administered to a living body. The diagnostically effective amount of the labelled compound of the invention to be administered before conducting the in-vivo method for the present invention is within a range of from 0.1 ng to 100 mg per kg body weight, preferably within a range of from 1 ng to 10 mg per kg body weight.

### EXAMPLES

All reactions involving air sensitive reagents or intermediates were performed under nitrogen and in anhydrous solvents. Magnesium sulfate was used as drying agent in the workup-procedures and solvents were evaporated under reduced pressure.

### Example 1

### Preparatory Example

### 1,4-Diazabicyclo[3.2.2]nonane (Intermediate Compound 1)

The title compound was prepared according to J. Med. Chem. 1993 36 2311-2320 (and according to the slightly modified method described below).

### 1,4-Diazabicyclo[3.2.2]nonane (Intermediate Compound 2)

To the solution of 1,4-diazabicyclo[3.2.2]nonan-3-one (15.8 g; 113 mmol) in absolute dioxane (130 ml) LiAlH₄ (4.9 g; 130 mmol) was added under argon. The mixture was refluxed for 6 h and then allowed to reach room temperature. To the reaction mixture water (5 ml in 10 ml of dioxane) was added by drops, the mixture was stirred for 0.5 hour and then filtered off via glass filter. The solvent was evaporated and the residue was distilled using Kugelrohr apparatus at 90° C. (0.1 mbar) to yield 1,4-diazabicyclo[3.2.2]nonane (11.1 g; 78%) as colourless hygroscopic material.

### 1,4-Diazabicyclo[3.2.2]nonan-3-one (Intermediate Compound 3)

To the solution of 3-quinuclidinone hydrochloride (45 g; 278 mmol) in 90 ml of water hydroxylamine hydrochloride (21 g; 302 mmol) and sodium acetate (CH₃COONa×3H₂O; 83 g; 610 mmol) were added, the mixture was stirred at 70° C. for 1 hour and then cooled to 0° C. The separated crystalline material was filtered off (without washing) and dried in vacuo to yield 40.0 g of oxime.

The 3-quinuclidinone oxime (40.0 g) was added during 2 hours by small portions to preheated to 120° C. polyphosphoric acid (190 g). The temperature of the solution during the reaction was kept at 130° C. After addition of all oxime the solution was stirred for 20 minutes at the same temperature, and was allowed to reach room temperature. The acidic mixture was neutralized by a solution of potassium carbonate (500 g in 300 ml of water), transferred into 2000 ml flask, diluted with 300 ml of water and extracted with chloroform (3×600 ml). The combined organic extracts were dried with sodium sulphate, the solvent evaporated and the solid residue dried up in vacuo to yield 30.0 g (77%) of the mixture of lactams.

Crystallization of the obtained mixture from 1,4-dioxane (220 ml) gave 15.8 g (40.5%) of 1,4-diazabicyclo[3.2.2]nonan-3-one as colourless large crystals with mp. 211-212° C.

### 2-(1,4-Diazabicyclo[3.2.2]nonan-4-yl)-5-(1-[¹¹C]methylpyrrol-2-yl)-1,3,4-oxadiazole (Compound 1)

Prepared from the corresponding precursor des-methyl precursor: 2-(1,4-diazabicyclo[3.2.2]nonan-4-yl)-5-(1H-pyrrol-2-yl)-1,3,4-oxadiazole by reaction with [¹¹C]methyl triflate.

The use of a [¹¹C]methyl-electrophile is not limited to the triflate, as an example [¹¹C]methyl iodide may also be used.

### Method A

### 2-(1,4-Diazabicyclo[3.2.2]nonan-4-yl)-5-(1H-pyrrol-2-yl)-1,3,4-oxadiazole Free Base (Intermediate Compound A1)

A mixture of 1,4-diazabicyclo[3.2.2]nonane (3.02 g, 23.9 mmol), 5-(1H-pyrrol-2-yl)-1,3,4-oxadiazole-2-thiol (5.0 g, 20.9 mmol) and 1-pentanol (50 ml) was stirred for 15 h. The mixture was solved in chloroform and was filtered through celite. The mixture was purified three times by silica gel chromatography, using chloroform, methanol and aqueous ammonia (89:10:1). The product was dried and evaporated. Yield 382 mg (6%). LC-ESI-HRMS of [M+H]+ shows 260.15096 Da. Calc. 260.15059 Da, dev. 1.4 ppm.

### Method B

### 5-(1H-pyrrol-2-yl)-1,3,4-oxadiazole-2-thiol (Intermediate Compound B1)

Potassium hydroxide (4.78 g, 85.3 mmol) was solved in methanol (125 ml). 1H-pyrrole-2-carbohydrazide (9.7 g, 77.5 mmol) was added and the mixture was stirred for 30 minutes. Carbon disulfide (14.7 g, 193.8 mmol) was added to the mixture followed by stirring at 65° C. for 15 h. Another equivalent of carbon disulfide (5.90 g, 77.5 mmol) was added followed by stirring at 65° C. for 4 days. Aqueous hydrochloric acid (1 M) was added in excess quantity, the mixture was stirred and filtered and washed with aqueous hydrochloric acid. Yield 10 g (77%).

### Method C

### 1H-pyrrole-2-carbohydrazide (Intermediate Compound C1)

Hydrazine monohydrate (31.0, 620 mmol) was added to a mixture of methyl 1H-pyrrole-2-carboxylate and methanol (100 ml) followed by stirring at 65° C. for 15 h. The mixture was evaporated and the product was isolated as a crystalline solid.

2-(1,4-Diazabicyclo[3.2.2]nonan-4-yl)-5-(1-methylpyrrol-2-yl)-1,3,4-oxadiazole fumaric acid salt (Intermediate Compound C2)

Prepared according to Methods A, B and C.

LC-ESI-HRMS of [M+H]+ shows 274.1671 Da. Calc. 274.16624 Da, dev. 3.1 ppm.

### Example 2

### Regional Time Activity Curve

This example illustrates the regional time activity curve (TAC) of the uptake and distribution of Compound 1 of the invention (i.e. 2-(1,4-Diazabicyclo[3.2.2]nonan-4-yl)-5-(1-[¹C]methylpyrrol-2-yl)-1,3,4-oxadiazole) in the pig brain.

Five Danish Landrace pigs were PET scanned in a high-resolution research tomography (HRRT) scanner after i.v. bolus injection of Compound 1 at t=0 mins. Points and connecting line indicate regional radioactive concentration normalized to injected dose per kg body weight, i.e. standardized uptake values (SUV).

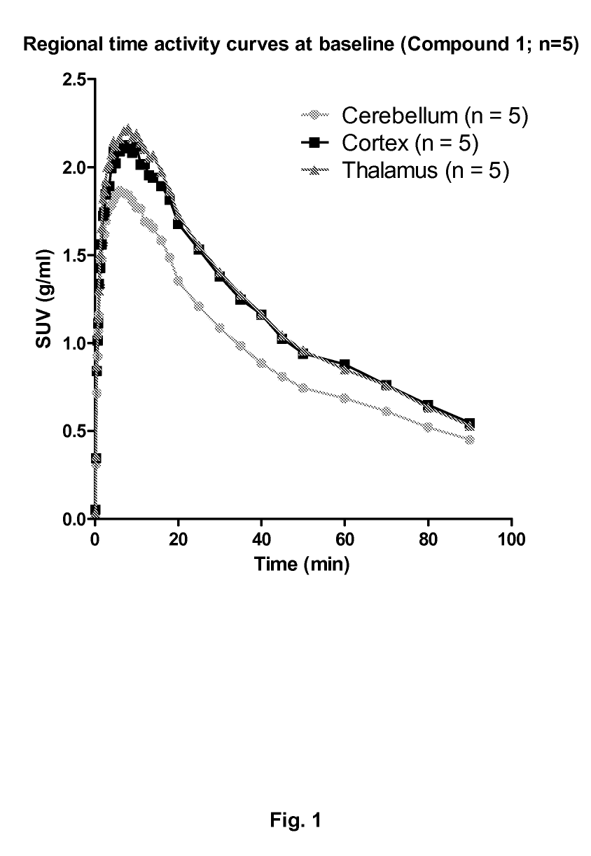

The results of this experiment are presented in FIG. 1.

### Example 3

### Summed PET Images

This example illustrates the summed PET images of Compound 1 of the invention (i.e. 2-(1,4-Diazabicyclo[3.2.2]nonan-4-yl)-5-(1-[¹¹C]methylpyrrol-2-yl)-1,3,4-oxadiazole) in the pig brain.

Representative images of one Danish Landrace pig after Compound 1 PET scanning at baseline (A) or after 10 mg/kg i.v. Compound 1 pre-treatment (B). Compound 1 pre-treatment was given 30 min before second scan. Images represent total activity summed from 0 to 90 minutes of PET scanning. Purple and blue colours represent low radioactivity signal, while green, red to white represent higher radioactivity signal. Higher radioactivity indicates higher receptor binding. Coronal, sagittal, and horizontal sections are shown from top to bottom row.

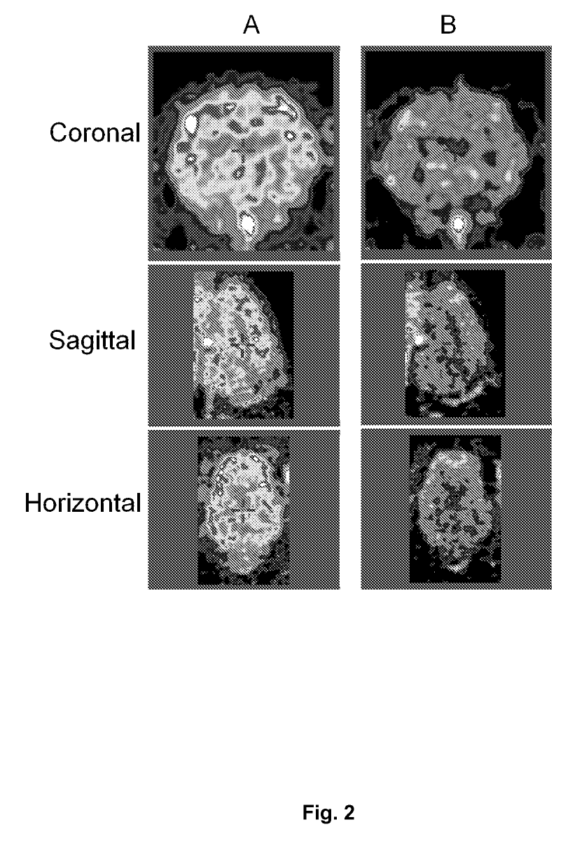

The results of this experiment are presented in FIG. 2.

### Example 4

### Distribution Volumes

In this example the distribution volumes (V_{T}) of Compound 1 of the invention (i.e. 2-(1,4-Diazabicyclo[3.2.2]nonan-4-yl)-5-(1-[¹¹C]methylpyrrol-2-yl)-1,3,4-oxadiazole) are compared to those of a selective nicotinic α7 acetylcholine receptor partial agonist known in the art, SSR180711 (i.e. 1,4-Diazabicyclo[3.2.2]nonane-4-carboxylic acid, 4-bromophenyl ester). The distribution volumes are determined in the pig brain at baseline, or following pre-treatment with either Compound 1 or SSR180711.

Five pigs were PET scanned at baseline with Compound 1 in the HRRT scanner. Subsequently, the pigs were administered either 10 mg/kg Compound 1 or SSR180711 (1 mg/kg or 10 mg/kg, i.v.), and 30 minutes or 4 hours later, the pigs were scanned a second time using the same PET protocol.

V_{T} is a measure of concentrations of Compound 1 in a given region relative to the plasma concentration. V_{T} was calculated by kinetic modelling using Logan plot linearization. All kinetic modelling was done in PMOD version 3.0 (PMOD technologies, Inc.). V_{T} is decreased following both Compound 1 and SSR180711, indicating that Compound 1 binds specifically and selectively to nicotinic α7 acetylcholine receptor in the pig brain.

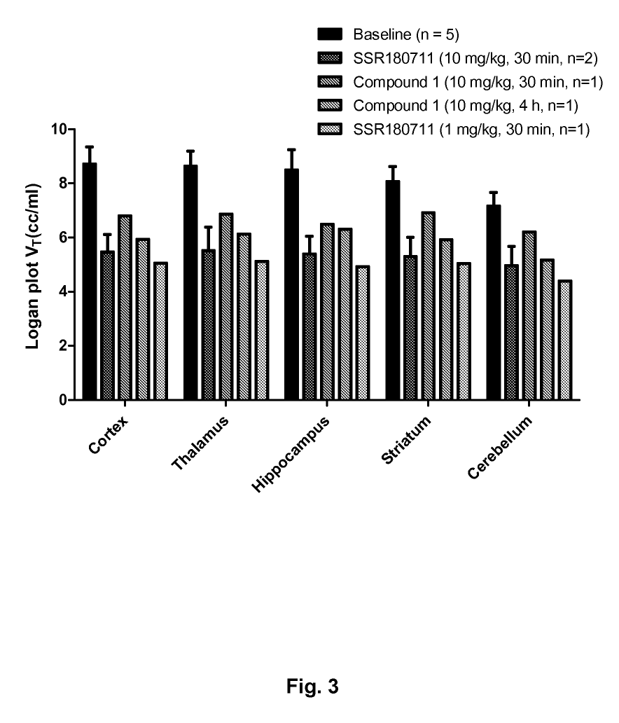

The results of this experiment are presented in FIG. 3, wherein Compound X=Compound 1.

### Example 5

### Receptor Occupancy

In this example Compound 1 of the invention (i.e. 2-(1,4-Diazabicyclo[3.2.2]nonan-4-yl)-5-(1-[¹C]methylpyrrol-2-yl)-1,3,4-oxadiazole) are compared to a selective nicotinic α7 acetylcholine receptor partial agonist known in the art, SSR180711 (i.e. 1,4-Diazabicyclo[3.2.2]nonane-4-carboxylic acid, 4-bromophenyl ester), in order to determine the level of displacement of the tracer and thereby calculating receptor occupancy

Compound 1 was given as an intravenous bolus injection to a total of five Danish Landrace pigs, and the pigs were subsequently scanned for 90 minutes in a high resolution research tomography (HRRT) PET scanner. Hereafter, the pigs were re-scanned after pre-treatment with cold ligand, either Compound 1 (10 mg/kg, iv) or SSR180711 (1 mg/kg or 10 mg/kg, iv).

The PET scans showed that Compound 1 entered into the pig brain, and the distribution was mainly found in the cerebral cortex and the thalamus as earlier reported. Pre-treatment with Compound 1 and SSR180711 lead to a decline in the distribution volumes (V_{T}) of Compound 1 as determined with logan plot kinetic modelling.

Furthermore, occupancy plotting of logan plot V_{T} showed that 10 mg/kg SSR180711 occupied 70% and 59% of nicotinic α7 acetylcholine receptor in the pig brain, respectively. Similarly, a lower dose of 1 mg/kg SSR180711 occupied 52% of the receptor. Pre-treatment with 10 mg/kg Compound 1 lead to occupancy of nicotinic α7 acetylcholine receptors of 81% when second scan was conducted 30 minutes after pre-treatment. Increasing this inter-scan interval to 4 hours decreased receptor occupancy to 43%.

1. A labelled pyrrolyl-oxadiazole-diazabicyclononane derivative represented by Formula I 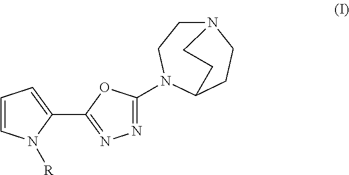
or a pharmaceutically acceptable salt thereof, wherein R represents a labelled C₁₋₆-alkyl group.

2. The labelled pyrrolyl-oxadiazole-diazabicyclononane derivative of claim 1, wherein the C₁₋₆-alkyl group is labelled by incorporation of one or more ¹¹C, ¹³C or ¹⁴C.

3. The labelled pyrrolyl-oxadiazole-diazabicyclononane derivative of claim 1, wherein R represents a labelled methyl or ethyl group.

4. The labelled pyrrolyl-oxadiazole-diazabicyclononane derivative of claim 1, which is
2-(1,4-Diazabicyclo[3.2.2]nonan-4-yl)-5-(1-[¹¹C]methylpyrrol-2-yl)-1,3,4-oxadiazole;
or a pharmaceutically acceptable salt thereof.

5. A pharmaceutical composition comprising a diagnostically effective amount of a labelled pyrrolyl-oxadiazole-diazabicyclononane derivative of claim 1, or a pharmaceutically acceptable addition salt thereof; together with at least one pharmaceutically acceptable carrier or diluent.

6. The labelled pyrrolyl-oxadiazole-diazabicyclononane derivative of claim 1, for use as a radiotracer to monitor in vivo and in vitro levels of nicotinic acetylcholine receptors, by way of non-invasive determination of the localisation of such receptors (neuroimaging).

7. The use according to claim 6, wherein the nicotinic acetylcholine receptor is of the nicotinic α7 receptor subtype.

8. A method for the non-invasive determination of the distribution of a tracer compound inside a whole, intact living animal or human body, using a physical detection method, wherein the tracer compound is a labelled pyrrolyl-oxadiazole-diazabicyclononane derivative according to claim 1, or a pharmaceutically acceptable salt thereof.

9. The method of claim 8, wherein the compound is labelled by incorporation of ¹¹C, ¹³C or ¹⁴C, and the isotope incorporation is measured by Positron Emission Tomography (PET) or Single Photon Emission Computed Tomography (SPECT).

10. The method of claim 9, wherein the compound is labelled by incorporation of ¹¹C, and the isotope incorporation is measured by Positron Emission Tomography (PET).

11. The method of claim 8, wherein the compound is 2-(1,4-diazabicyclo[3.2.2]nonan-4-yl)-5-(1-[¹¹C]methylpyrrol-2-yl)-1,3,4-oxadiazole.
